(19) United States
(12) Patent Application Publication
(10) Pub. No.: US 2026/0240499 A1
(43) Pub. Date: Aug. 20, 2026

(54) AI-Powered Multi-Modal Health Monitoring System and Method of Use

(71) Applicant: Adam Denoto, Brook Park, OH (US)

(72) Inventor: Adam Denoto, Brook Park, OH (US)

(21) Appl. No.: 19/411,910

(22) Filed: Dec. 8, 2025

Related U.S. Application Data
(60) Provisional application No. 63/760,702, filed on Feb. 20, 2025.

(51) Int. Cl. A61B 5/00; A61B 5/0205; A61B 5/20; A61M 11/04; G16H 40/40; G16H 50/20

(52) U.S. Cl. CPC A61B 5/6892; A61B 5/0022; A61B 5/02055; A61B 5/207; A61B 5/7267; A61B 5/7435; A61M 11/041; G16H 40/40; G16H 50/20

(57) ABSTRACT

A ‘smart’ healthcare bed system that integrates a plurality of diagnostic modules with artificial intelligence to perform real-time health monitoring, diagnosis, and telemedicine is disclosed. The bed includes a body scanning module using infrared, ultrasonic, or optical sensors, a urine analysis module, a vital signs monitoring suite, and a blood analysis unit for non-invasive biomarker detection. An AI-powered diagnostic module analyzes the recorded data, generates diagnoses, and provides personalized health recommendations. A user interface displays diagnostic results and supports patient interaction, while a wireless communication module enables secure connectivity to external systems. A remote server system communicates with the bed to manage encrypted patient data, update AI models via over-the-air updates, and facilitates telemedicine by matching patients with healthcare providers. The server includes databases for patients, healthcare providers, and diagnostic learning, ensuring adaptive care and model training.

### CROSS-REFERENCE TO RELATED APPLICATION

The present application claims priority to, and the benefit of, U.S. Provisional Application No. 63/760,702 which was filed on February 20, 2025 and is incorporated herein by reference in its entirety.

### FIELD OF THE INVENTION

The present invention generally relates to medical diagnostic systems and healthcare technologies. More specifically, the present invention relates to an AI-powered ‘smart’ healthcare bed system configured to autonomously monitor, diagnose, and manage a user’s health status in real-time. The system comprises a patient-supporting bed structure embedded with integrated diagnostic modules including a body scanning unit, urine analysis system, vital signs monitoring suite, and blood analysis unit, all operatively coupled to an AI diagnostic processor. The system further includes a user interface for visual and audio feedback, and a wireless communication module for remote connectivity. The AI diagnostic module utilizes machine learning algorithms to analyze physiological and biochemical data and generate personalized diagnoses, risk scores, and health recommendations. Accordingly, the present disclosure makes specific reference thereto. Nonetheless, it is to be appreciated that aspects of the present invention are also equally applicable to other like applications, devices, and methods of manufacture.

### BACKGROUND

By way of background, conventional healthcare system has faced challenges in the recent years. One challenge involves the potential for conflicts of interest, where healthcare providers may be influenced by pharmaceutical companies or insurance-driven incentives that prioritize profit generation over patient-centered care. Incentives can compromise diagnostic objectivity and may result in suboptimal treatment decisions that do not fully align with the patient's best interests.

Additionally, patient data privacy remains an issue in conventional healthcare systems. Despite regulations to safeguard personal health information, many cases of data breaches and unauthorized access to medical records have raised questions about the effectiveness of existing data protection mechanisms. The lack of robust, user-controlled data security in many healthcare infrastructures continues to be a major source of concern.

Furthermore, home-based healthcare, particularly for individuals with chronic illnesses, disabilities, or limited mobility, can be complex and burdensome. Caregivers often struggle with a lack of real-time data, inconsistent monitoring, and limited diagnostic tools, leading to delayed interventions and added stress. Accordingly, there is a growing need for a secure, accessible, and intelligent healthcare solution that can deliver unbiased medical analysis, protect patient privacy, and enable accurate, real-time diagnostics.

Therefore, there exists a long-felt and urgent need in the healthcare industry for a diagnostic and treatment system that is not influenced by profit-driven motives. There exists a long-standing need for a solution that combines multiple diagnostic technologies into a single platform to provide real-time medical assessment without the need for constant clinical intervention. Further, there is a critical need for an in-home healthcare system that maintains strict data privacy while offering secure telemedicine and remote care capabilities. Additionally, there is a need for a healthcare device that features an integrated AI that is trained on medical data to accurately diagnose medical conditions. Finally, there exists a need for a ‘smart’ healthcare bed that autonomously monitors a patient’s health, provides AI-driven diagnostic support, and facilitates secure communication with healthcare professionals.

The subject matter disclosed and claimed herein, in one embodiment, comprises a ‘smart’ healthcare bed system incorporating integrated diagnostic sensors, AI-powered analytics, a secure communication interface, and a patient-friendly display. The bed system includes a frame that supports a patient surface embedded with modules for scanning the body, analyzing urine, monitoring vital signs, and performing blood analysis. A central AI-powered diagnostic module is operably coupled to each of these subsystems and configured to process real-time physiological data collected from a user lying on the bed. Based on this data, the AI module generates diagnosis output, assesses medical risk levels, and provides prescriptions or health recommendations. A display device is positioned on the bed to visually and audibly communicate health information to the user. The system also includes a wireless communication module that enables secure data exchange with external servers, cloud systems, and telemedicine platforms.

In one embodiment, the smart bed includes a body scanning module configured to perform both surface and subdermal imaging using infrared, ultrasonic, or optical sensors to detect musculoskeletal conditions, skin abnormalities, or other anomalies. A urine analysis module is embedded in the bed surface and is activated upon detection of urination. The urine module includes microfluidic biosensors configured to test for glucose, ketones, protein, nitrites, pH, leukocytes, and more, thereby offering metabolic and infection-related insights. A vital signs suite comprising ECG, temperature, respiratory, and blood pressure sensors provides continuous monitoring of the user’s condition. Additionally, a blood analysis unit facilitates non-invasive or minimally invasive biomarker testing, including glucose and hemoglobin levels, using optical or needle-free technologies. The AI-powered diagnostic module is configured to locally process sensor data and generate diagnostic reports. In some embodiments, the diagnostic model is trained on large datasets of de-identified patient records and clinical guidelines and can be updated through over-the-air (OTA) updates.

In this manner, the ‘smart’ healthcare bed system of the present invention overcomes longstanding deficiencies in traditional medical systems by enabling unbiased, AI-driven diagnostics, maintaining strict user privacy, and enhancing access to professional care without leaving home. The system addresses the inefficiencies of fragmented diagnostic tools by consolidating them into a single, intelligent bed platform. The system integrates local AI processing, real-time health monitoring, and telehealth services to provide users with continuous, accurate, and responsive healthcare support. The system also maintains privacy through data encryption to comply with strict medical privacy laws and focuses on medical care for individuals rather than profit-driven medical decisions.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a ‘smart’ healthcare bed system. The ‘smart’ healthcare bed system comprises a body scanning module disposed within or adjacent to the patient support frame. The body scanning module is configured to perform surface and subsurface imaging of the user's body using one or more imaging techniques selected from the group consisting of infrared, ultrasonic, and optical sensing. The system also includes a urine analysis module that is integrated into the patient support frame and configured to automatically (i.e., autonomously) collect and analyze urine samples from the user to detect biomarkers indicative of one or more medical conditions. A vital signs monitoring suite is embedded within the support frame and is configured to measure at least heart rate, blood pressure, body temperature, respiratory rate, and blood oxygen saturation. Additionally, a blood analysis unit is included in the bed and is configured to perform non-invasive or minimally invasive blood analysis for parameters such as glucose and hemoglobin levels.

In another embodiment, the bed system includes an AI-powered diagnostic module configured to receive real-time data from the modules. The AI-powered diagnostic module stores one or more machine learning models trained on historical medical data and is configured to generate a diagnosis and provide personalized health insights and prescription recommendations. A display device is coupled to the AI-powered diagnostic module and is configured to present diagnostic results and insights to the user. Finally, a wireless communication module is provided and configured to transmit and receive encrypted health data to and from external servers or healthcare systems.

In still another embodiment, a remote server system works with the ‘smart’ healthcare bed. The server includes a patient database configured to store anonymized patient health records including biometric data, diagnostic history, treatment compliance records, and AI-generated insights received from the smart bed. The server system further includes a doctor database that stores healthcare provider profiles, availability schedules, certifications, and specialties. A diagnostic learning database is also included and configured to store historical and real-time medical datasets, machine learning models, diagnostic rules, and clinical decision support logic. The server is configured to analyze the received diagnostic data, update the diagnostic models based on aggregated learning, and transmit improved or retrained models back to the ‘smart’ healthcare bed system for deployment via over-the-air (OTA) updates.

In one embodiment, a method for performing automated medical diagnosis using a ‘smart’ healthcare bed system is disclosed and comprises acquiring physiological and biochemical data from a user using sensors embedded within the bed. The data includes body scan images, urine biomarkers, vital signs such as heart rate and blood pressure, and blood composition metrics. The acquired data is transmitted to an AI-powered diagnostic module embedded within the bed. The AI-powered diagnostic module processes the data using a machine learning model trained on historical health records and biometric sensor data. Based on the processing, the module generates a medical diagnosis and provides personalized health insights. The system then displays this information on a user interface coupled to the ‘smart’ healthcare bed, including diagnostic results and recommended prescriptions.

In yet another embodiment, a method for training an artificial intelligence diagnostic model for use in a ‘smart’ healthcare bed is disclosed and includes storing a plurality of anonymized medical records. The records contain biometric sensor data, diagnostic outcomes, and treatment histories, and are stored in compliance with applicable data protection regulations. The stored records are labeled with verified diagnoses that are derived from clinical data and standardized diagnostic criteria. The labeled records are then used to train a machine learning model capable of identifying relationships between sensor data patterns and known medical conditions. Upon completion of training, the model is deployed to the diagnostic module of a ‘smart’ healthcare bed for use in generating real-time diagnoses and health insights during patient use.

Numerous benefits and advantages of this invention will become apparent to those skilled in the art to which it pertains upon reading and understanding of the following detailed specification.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a block diagram of an exemplary embodiment of ‘smart’ healthcare bed in accordance with the disclosed structure;

FIG. 2 illustrates a block-level schematic diagram of the communication of the ‘smart’ healthcare bed with external systems in accordance with one embodiment of the present invention;

FIG. 3 illustrates an exemplary graphical representation of a diagnostic user interface displayed by the display device of the smart bed system in accordance with the disclosed structure;

FIG. 4 illustrates a high-level flowchart depicting the diagnostic operation workflow of the AI-powered smart bed system of the present invention in accordance with the disclosed structure;

FIG. 5 illustrates a flowchart depicting the process of training the AI diagnostic model used in the smart bed system in accordance with one embodiment of the present invention;

FIG. 6 illustrates a flowchart depicting the process for initiating and conducting a telemedicine session using the ‘smart’ healthcare bed system of the present invention in accordance with the disclosed structure;

FIG. 7 illustrates a perspective view of exemplary physical structure of the AI-powered smart bed system of the present invention in accordance with the disclosed structure; and

FIG. 8 illustrates a perspective view of another embodiment of the AI-powered smart bed system of the present invention in accordance with the disclosed structure.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there exists a long-felt and urgent need in the healthcare industry for a diagnostic and treatment system that is not influenced by profit-driven motives. There exists a long-standing need for a solution that combines multiple diagnostic technologies into a single platform to provide real-time medical assessment without the need for constant clinical intervention. Further, there is a critical need for an in-home healthcare system that maintains strict data privacy while offering secure telemedicine and remote care capabilities. Additionally, there is a need for a healthcare device that features an integrated AI that is trained on medical data to accurately diagnose medical conditions. Finally, there exists a need for a ‘smart’ healthcare bed that autonomously monitors a patient’s health, provides AI-driven diagnostic support, and facilitates secure communication with healthcare professionals.

The present invention, in one exemplary embodiment, is a method for performing automated medical diagnosis using a ‘smart’ healthcare bed system and comprises acquiring physiological and biochemical data from a user using sensors embedded within the bed. The data includes body scan images, urine biomarkers, vital signs such as heart rate and blood pressure, and blood composition metrics. The acquired data is transmitted to an AI-powered diagnostic module embedded within the bed. The AI-powered diagnostic module processes the data using a machine learning model trained on historical health records and biometric sensor data. Based on the processing, the module generates a medical diagnosis and provides personalized health insights. The system then displays this information on a user interface coupled to the ‘smart’ healthcare bed, including diagnostic results and recommended prescriptions.

Reference will now be made in detail to the present preferred embodiments of the invention, examples of which are illustrated in the accompanying drawings. Wherever possible, the same reference numerals are used in the drawings and the description to refer to the same or like parts.

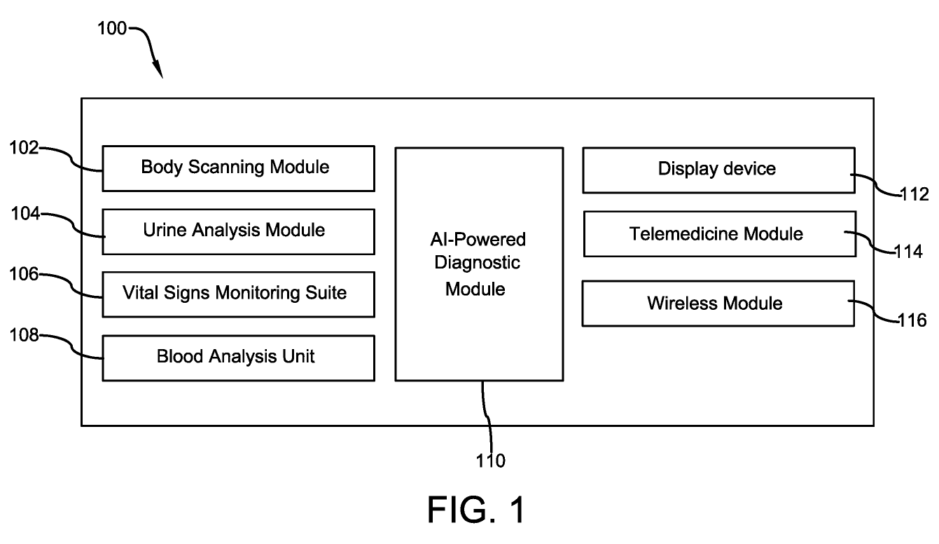

Referring initially to the drawings, FIG. 1 illustrates a block diagram of an exemplary embodiment of ‘smart’ healthcare bed in accordance with the disclosed structure. The ‘smart’ healthcare bed 100 of the present invention is designed as an advanced and Artificial Intelligence (AI) powered bed to monitor, diagnose, and provide health advice and prescriptions in real time. More specifically, the ‘smart’ healthcare bed 100 includes a body scanning module 102 configured to perform full-body surface and subsurface scans using imaging techniques such as infrared, ultrasonic, or optical sensors. The body scanning module 102 may include one or more of infrared, ultrasonic, or optical sensors and may detect musculoskeletal anomalies, skin conditions, or surface lesions.

A urine analysis module 104 is configured to automatically (i.e., autonomously) collect and analyze urine samples from a user lying on the ‘smart’ healthcare bed 100 to detect biomarkers relevant to kidney function, infections, hydration status, and metabolic disorders. The urine analysis module 104 can be embedded in the bed surface and may be activated using a pressure sensor when urination is detected. The urine analysis module 104 comprises microfluidic biosensors for at least glucose, ketones, protein, pH, nitrites, leukocytes, bilirubin, urobilinogen, and more.

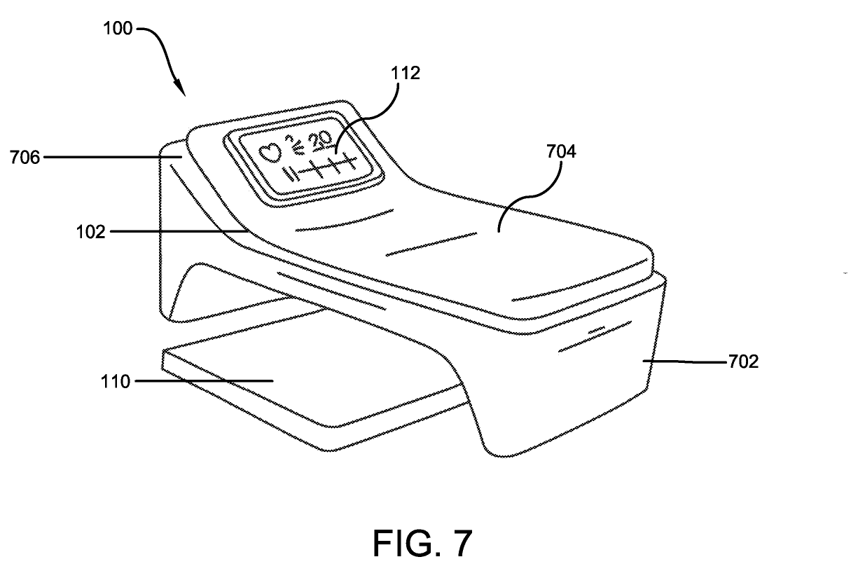

A vital signs monitoring suite 106 is embedded in the ‘smart’ healthcare bed 100 and includes a plurality of vital signs monitors including but not limited to ECG sensor, body temperature sensor, respiratory sensor, blood pressure sensor, and SpO2 sensor as described in FIG. 7. The suite 106 may have a nebulizer to provide fast and targeted treatment of lung and airway conditions. The nebulizer can be used for patients suffering from at least one of asthma, chronic obstructive pulmonary disease (COPD), bronchitis, cystic fibrosis, respiratory infections, and severe allergic reactions affecting breathing.

A blood analysis unit 108 is included in the ‘smart’ healthcare bed 100 and facilitates non-invasive or minimally invasive blood analysis. The blood analysis is preferably done through optical sensors and includes glucose levels, hemoglobin, electrolytes, and other blood biomarkers.

An AI powered diagnostic module 110 is in the form of an AI-powered processor. The AI powered diagnostic module 110 receives real-time physiological, biochemical, and behavioral data from all diagnostic modules 102, 104, 106, 108 and provides at least diagnosis, personalized health insights, and severity ratings. The AI powered diagnostic module 110 may include an onboard processor with AI acceleration, memory for logs and machine learning models, and a plurality of data interfaces for communication with different modules/sensors.

Preferably, the AI powered diagnostic module 110 performs local processing, however, can communicate with a remote server for advanced processing of data of diagnostic modules as described later in the disclosure. The AI powered diagnostic module 110 is trained on de-identified historical health records, biometric sensor data, EHRs, and research-based clinical guidelines and the AI models are updated periodically via OTA (Over-The-Air) updates.

The ‘smart’ healthcare bed 100 includes a display device 112 which is preferably a touch screen device and is coupled to the AI powered diagnostic module 110. The display device 112 displays the vital signs and diagnostic information as visual feedback to users about a patient/user lying on the ‘smart’ healthcare bed 100. In some embodiments, the display device 112 can also provide voice-based feedback. The display device 112 can also provide medication recommendations and AI doctor interaction with the patient.

Preferably, the display device 112 is embedded at the headboard or bedside console of the smart bed 100 and can use LCD, OLED, or any other display technology. The display device 112 may provide safe readings of different modules in a first color such as Green color and abnormal readings (i.e., above or below a corresponding threshold) in a second color which is different from the first color. As a non-limiting example, when vitals of a user indicate abnormal levels such as high glucose, the display device 112 may notify the user visually and audibly.

A telemedicine module 114 enables a user/patient to initiate a remote consultation with a licensed healthcare provider. The telemedicine module 114 is configured to share real-time data from the AI powered diagnostic module 110 and support encrypted video/audio communications for a secure medical information transmission (i.e., transmitting and receiving) from the ‘smart’ healthcare bed 100.

A wireless communication module 116 is configured to provide wireless communication channel for secure communication with external systems and devices such as a telepath network or a server. The wireless communication module 116 may support one or more from Wi-Fi, Bluetooth, LTE/5G, Zigbee, or any other short-range or long-range wireless communication technology. The wireless communication module 116 enables the ‘smart’ healthcare bed 100 to synchronize with the external devices, electronic health records (EHR) integration, and update AI machine learning models using over-the-air firmware updates.

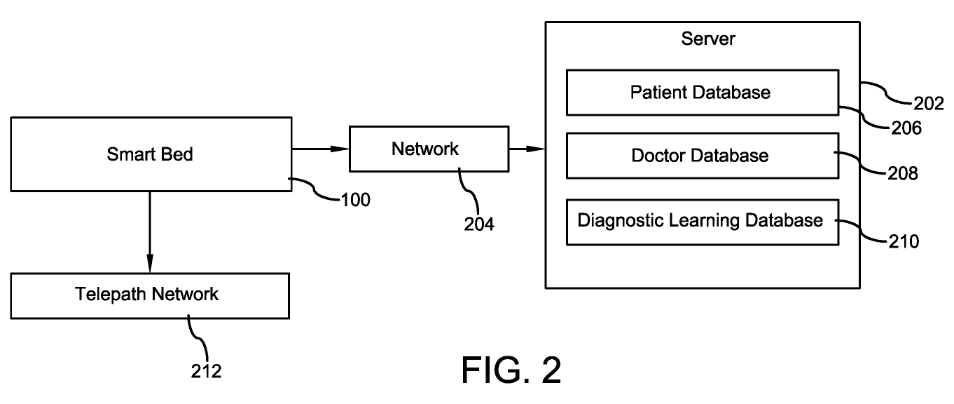

FIG. 2 illustrates a block-level schematic diagram of the communication of the ‘smart’ healthcare bed with external systems in accordance with one embodiment of the present invention. The ‘smart’ healthcare bed 100 using the wireless communication module 116 (FIG. 1) can connect to at least one external (i.e., backend) or remote server 202 and a telepath network 212 via a wireless network 204. The remote server 202 is a cloud-based AI training server and includes a plurality of modules to enable AI training model updates, cloud-based analysis of the data captured by different sensors of the smart bed 100 and OTA firmware upgrades.

The network 204 can be any suitable data communication infrastructure such as Wi-Fi, LTE/5G, internet backbone, and facilitates bi-directional data exchange between the smart bed 100 and the backend server system 202.

The remote server 202 includes a patient database 206 to store anonymized and encrypted health records, vital signs, diagnostic history, and treatment compliance data of different users using the smart bed 100. The patient database 206 is used for providing personalized care and enhanced diagnosis for the patients.

A doctor database 208 stores information about licensed healthcare providers, including availability, specialties, and certifications. The remote server 202 may use both the patient database 206 and the doctor database 208 to recommend one or more medical professionals or licensed healthcare providers for a patient for telemedicine sessions.

A diagnostic learning database 210 is configured to store real-time medical case data, outcomes, machine learning models, and rule-based decision systems. The database 210 can be used by AI powered diagnostic module 110 to update and validate diagnostic algorithms used by the ‘smart’ healthcare bed 100.

A telepath network 212 provides a dedicated communication infrastructure to enable real-time interaction between patients and medical professionals or licensed healthcare providers. The telepath network 212 may use a secure API such as WebRTC or video conferencing APIs to connect the patient at the smart bed 100 to a remote healthcare provider.

The system of the invention is designed to provide secure, at-home medical evaluation, enhance early detection of medical conditions, reduce dependence on clinical visits, and enable real-time telemedicine through encrypted data sharing with licensed healthcare providers.

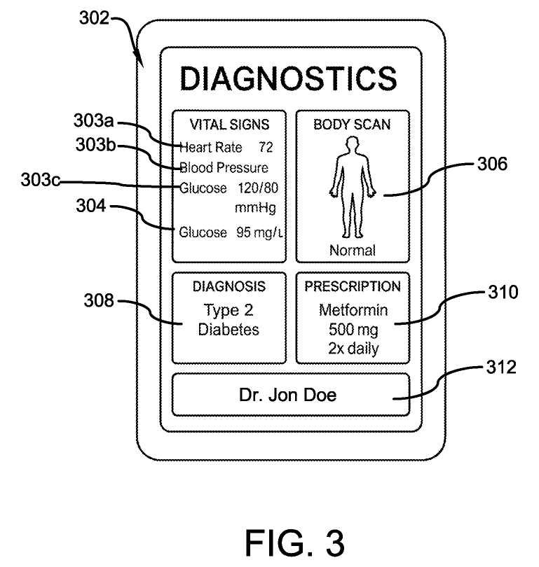

FIG. 3 illustrates an exemplary graphical representation of a diagnostic user interface displayed by the display device of the smart bed system in accordance with the disclosed structure. The user interface 302 includes a plurality of panels for displaying various diagnostic categories of a user. A vital signs panel 304 displays critical vital signs captured by integrated sensor modules of the smart bed 100. The vital signs panel 304 displays at least heart rate metrics 303a, blood pressure metrics 303b, and blood glucose metrics 303c. The values are displayed in real-time and are updated continuously. The vital signs panel 304 may be coded in different colors to visually indicate the risk levels based on the detected vital signs values.

A body scan panel 306 displays a visual representation of a patient's body based on imaging techniques embedded in the smart bed 100. The body scan panel 306 provides information and insights about musculoskeletal or dermatological conditions and abnormal body regions of the patient as a visual feedback.

A diagnosis panel 308 displays the medical interpretation (i.e., condition) determined by the AI-powered diagnostic module 110 of the ‘smart’ healthcare bed system 100. The diagnosis panel 308 can also provide a summary of the medical condition and associated severity level. In some embodiments, the diagnosis panel 308 may provide AI confidence score of the determined medical condition.

A prescription panel 310 displays the recommended treatment generated automatically (i.e., autonomously) by the AI-powered diagnostic module 110 based on the diagnosis. It should be noted that the treatment and diagnosis are generated using the diagnostic learning database 210 (FIG. 2) and the local processing and training models of the AI-powered diagnostic module 110.

The user interface 302 also displays the name of an assigned or consulting doctor 312 (i.e., Dr. Jon Doe) in the case of a telemedicine session or review. The doctor can be automatically (i.e., autonomously) selected by the bed 100 or remote server 202 based on the profile of the patient and diagnosis by the smart bed 100.

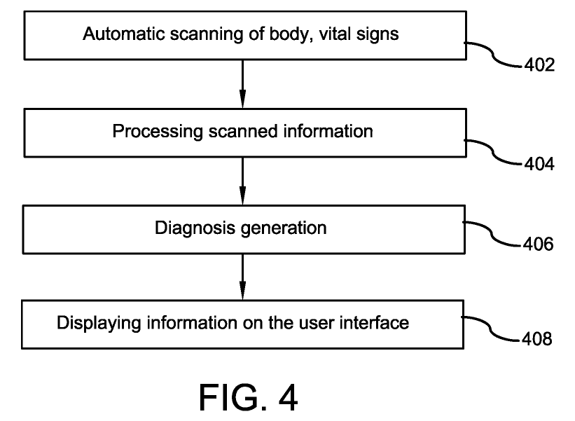

FIG. 4 illustrates a high-level flowchart depicting the diagnostic operation workflow of the AI-powered smart bed system 100 of the present invention in accordance with the disclosed structure. Initially, the smart bed 100 performs the automatic or autonomous scanning of the user's body and collection of vital signs (Step 402). The step is initiated when the user lies on the bed or manually activates a scan session and vital signs such as heart rate, blood pressure, respiration rate, blood glucose, and oxygen saturation are collected.

The data acquired in the above step is passed to the AI-powered diagnostic module 110 for processing and analysis (Step 404). The processing can include filtering noise, normalizing data across different formats and sampling rates, AI model inference, and more.

In the next step, the AI-powered diagnostic module 110 using the machine learning models trained on medical datasets, predicts medical conditions such as diabetes, infection, hypertension of the patient (Step 406). This step may also refer to the patient database 206 for historical data, sleep/activity patterns, or previous diagnoses.

Finally, the diagnostic results are displayed on the display device 112 (Step 408). The display device 112 may display various vital signs, diagnosis, recommended prescriptions, and more as illustrated in FIG. 3.

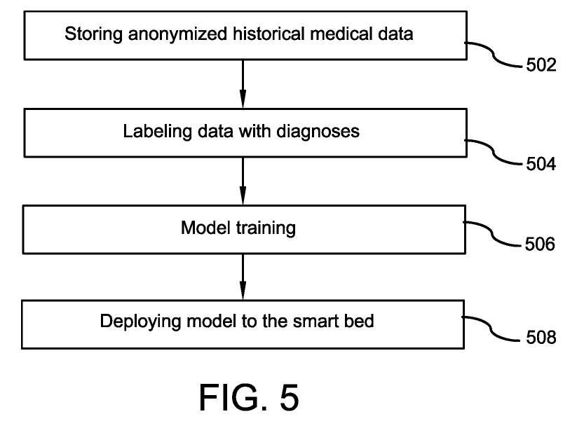

FIG. 5 illustrates a flowchart depicting the process of training the AI diagnostic model used in the smart bed system in accordance with one embodiment of the present invention. The training of the AI diagnostic models helps the smart bed 100 with the latest clinical insights, improved detection accuracy over time, and diagnostic capabilities across diverse patients.

Initially, anonymized medical datasets are stored locally in the smart bed system 100 and in the diagnostic learning database 210 (Step 502). The anonymized medical datasets may include at least past-patients health records, sensor-collected data, previous diagnostic outcomes and clinical notes. It should be noted that the data is stored in compliance with medical data privacy laws such as HIPAA, GDPR and does not contain any personally identifiable information (PII).

The stored data is then labeled with verified diagnostic outcomes (Step 504). The labels may be derived from clinical records from certified healthcare providers, medical imaging reports, diagnoses based on standardized clinical criteria, and more. The labeling enables the AI-powered diagnostic module 110 to associate specific sensor data patterns with known medical conditions, creating a supervised learning dataset.

Then, one or more machine learning algorithms of the AI-powered diagnostic module 110 are applied to the labeled dataset to create predictive models (Step 506). The model training may include medical feature extraction and selection along with cross-validation and performance evaluation for accuracy and recall of the data.

Then, the trained model is deployed to the AI-powered diagnostic module 110 embedded within the smart bed 100 (Step 508). The trained model may be deployed using one or more from over- the-air (OTA) software update, secure cloud-to-device communication, and local installation during setup or maintenance of the smart bed system 100.

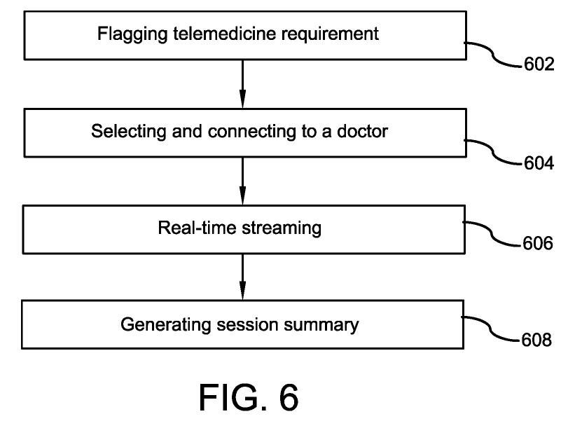

FIG. 6 illustrates a flowchart depicting the process for initiating and conducting a telemedicine session using the ‘smart’ healthcare bed system of the present invention in accordance with the disclosed structure. Initially, a requirement for a telemedicine session is automatically (i.e., autonomously) or manually flagged (Step 602). A requirement may be flagged when the AI diagnostic module 110 detects a condition that requires medical verification, a health threshold or anomaly is exceeded, or when the patient explicitly requests a doctor consultation via the user interface. Then, the smart bed 100 along with the remote server 202 identifies available, qualified doctors from the doctor database 208 and may match based on specialty, availability, and prior engagement with the patient (Step 604). The patient can be connected to the doctor using audio, video, or text-based conferencing.

In the next step, the bed system 100 enables real-time bi-directional streaming between the patient and the doctor (Step 606) and may include at least live video/audio communication and synchronized display of diagnostic results (i.e., vitals, scan images, AI diagnosis). The streaming is performed via the wireless communication module 116, using secure network protocols to maintain patient privacy and medical data compliance. Finally, at the conclusion of the telemedicine session, the bed system 100 automatically (i.e., autonomously) generates a session summary (Step 608) which may include diagnosis confirmed or updated by the physician, prescribed medication, timestamp, notes from the telemedicine session, and video recording.

FIG. 7 illustrates a perspective view of exemplary physical structure of the AI-powered smart bed system of the present invention in accordance with the disclosed structure. The bed system 100 includes a frame 702 that contains and supports all internal electronic and diagnostic modules as described in FIG. 1. The frame 702 may be fabricated from medical-grade composite materials or molded polymer for durability and hygiene. A patient surface 704 is in the form of a mattress zone on which the patient rests. The surface may be embedded with a plurality of modules as described in FIG. 1 for automatic sample collection. An adjustable head component 706 is configured to be tilted or elevated for comfort, diagnostics, or therapeutic positioning and can be mechanically or electronically controlled.

The display device 112 is embedded in the bed head area 706 for displaying diagnostic information to the user. The display device 112 provides visual dashboards of vital signs and AI-generated diagnoses, prescription instructions, controls for initiating scans or telemedicine sessions, and other information as described in FIG. 3. The AI-powered diagnostic module 110 is coupled to the different sensors and modules of the bed system 100 and conducts fluid analysis of urine, blood, and other bodily fluids, and vital signs acquisition and processing.

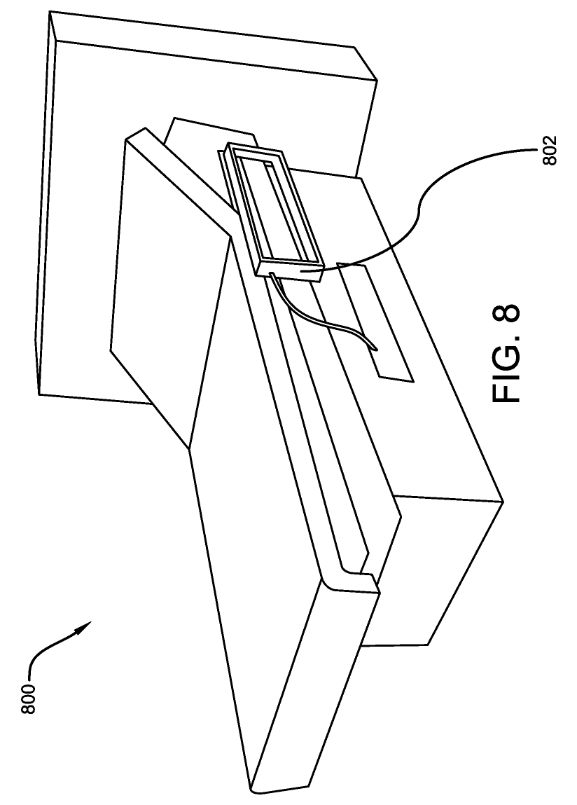

FIG. 8 illustrates a perspective view of another embodiment of the AI-powered smart bed system of the present invention in accordance with the disclosed structure. It should be noted that the AI-powered smart bed system can be designed in various sizes and shapes to accommodate requirements of different users. As illustrated, the smart bed system 800 is designed to use in a medical facility such as a hospital wherein the bed system 800 can be coupled with external medical tools 802 for enhancing utility of the bed system 800.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “AI-powered ‘smart’ healthcare bed system”, “‘smart’ healthcare bed system”, “AI-powered multi-modal health monitoring system”, and “‘smart’ healthcare bed” are interchangeable and refer to the AI-powered multi-modal health monitoring system 100, 800 of the present invention.

Notwithstanding the forgoing, the AI-powered multi-modal health monitoring system 100, 800 of the present invention can be of any suitable configuration as is known in the art without affecting the overall concept of the invention, provided that it accomplishes the above stated objectives. One of ordinary skill in the art will appreciate that the AI-powered multi-modal health monitoring system 100, 800 as shown in the FIGS. are for illustrative purposes only, and that many other configurations of the AI-powered multi-modal health monitoring system 100, 800 are well within the scope of the present disclosure. Although the dimensions of the AI-powered multi-modal health monitoring system 100, 800 are important design parameters for user convenience, the AI-powered multi-modal health monitoring system 100, 800 may be of any size that ensures optimal performance during use and/or that suits the user’s needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. An Artificial Intelligence (AI) healthcare bed comprising:
an AI powered diagnostic module;
a body scanning module;
a urine analysis module;
a vital signs monitoring suite; and
a blood analysis module;
wherein said body scanning module configured to perform full-body surface and subsurface scans using imaging sensors selected from the group consisting of infrared sensors, ultrasonic sensors, and optical sensors;
wherein said urine analysis module configured to autonomously collect and analyze urine samples from a user lying on the AI healthcare bed to detect biomarkers selected from the group consisting of kidney function, infections, hydration status, and metabolic disorders;
wherein said vital signs monitoring suite comprising one or more sensors selected from the group consisting of an ECG sensor, a body temperature sensor, a respiratory sensor, a blood pressure sensor, an SpO2 sensor;
wherein said blood analysis unit comprising optical sensors for monitoring glucose levels, hemoglobins, electrolytes, and blood biomarkers; and
further wherein said AI powered diagnostic module comprising an AI-powered processor for receiving in real-time information selected from the group consisting of physiological data, biochemical data, and behavioral data from one or more of said body scanning module, said urine analysis module, said vital signs monitoring suite, and said blood analysis unit.

2. The Artificial Intelligence (AI) healthcare bed of claim 1, wherein said vital signs monitoring suite further comprising a nebulizer to provide treatment of lung and airway conditions to patients suffering from at least one of asthma, chronic obstructive pulmonary disease (COPD), bronchitis, cystic fibrosis, respiratory infections, and severe allergic reactions affecting breathing.

3. The Artificial Intelligence (AI) healthcare bed of claim 1 further comprising a display device comprising a touch screen device coupled to said AI powered diagnostic module, wherein said display device displays vital signs as visual feedback about a user lying on the AI healthcare bed.

4. The Artificial Intelligence (AI) healthcare bed of claim 1 further comprising a telemedicine module for a user to initiate a remote consultation with a licensed healthcare provider, wherein said telemedicine module is configured to share real-time data from said AI powered diagnostic module.

5. The Artificial Intelligence (AI) healthcare bed of claim 4 further comprising a wireless communication module configured to provide bi-directional data exchange with an external device selected from the group consisting of a telepath network, a remote server, and an electronic health record (EHR).

6. The Artificial Intelligence (AI) healthcare bed of claim 5, wherein said remote server is a cloud-based AI training server to enable AI training model updates.

7. The Artificial Intelligence (AI) healthcare bed of claim 5, wherein said remote server comprises a patient database to store anonymized information selected from the group consisting of encrypted health records, vital signs, diagnostic history, and treatment compliance data of different users received from the AI healthcare bed.

8. The Artificial Intelligence (AI) healthcare bed of claim 7, further comprising a doctor database for storing information selected from the group consisting of licensed healthcare providers, provider availability, provider specialties, and provider certifications, wherein said remote server uses said patient database and said doctor database to recommend one or more said licensed healthcare providers for the user to receive a telemedicine session.

9. The Artificial Intelligence (AI) healthcare bed of claim 1 further comprising a diagnostic learning database configured to store real-time medical information selected from the group consisting of case data, outcomes, machine learning models, and rule-based decision systems, wherein said diagnostic learning database connected to said AI powered diagnostic module to update and validate diagnostic algorithms used by the AI healthcare bed.

10. The Artificial Intelligence (AI) healthcare bed of claim 1 further comprising a diagnostic user interface comprising a display device connected with the AI healthcare bed, wherein said diagnostic user interface comprising a plurality of display panels for displaying diagnostic categories of a user including a vital signs panel for displaying vital signs captured by said vital signs monitoring suite, further wherein said vital signs selected from the group consisting of heart rate metrics, blood pressure metrics, and blood glucose metrics displayed in real-time and updated continuously.

11. The Artificial Intelligence (AI) healthcare bed of claim 1 further comprising a body scan panel for displaying a visual representation of the user’s body based on body scans from said body scanning module.

12. The Artificial Intelligence (AI) healthcare bed of claim 11 further comprising a diagnosis panel for displaying a medical interpretation determined by said AI-powered diagnostic module of the AI healthcare bed, wherein said diagnosis panel provides a summary of said medical interpretation and an associated severity level of said medical interpretation.

13. The Artificial Intelligence (AI) healthcare bed of claim 12 further comprising a prescription panel for displaying a recommended treatment generated by said AI-powered diagnostic module based on said medical interpretation.

14. The Artificial Intelligence (AI) healthcare bed of claim 1, wherein said urine analysis module embedded in the AI healthcare bed and activated using a pressure sensor when urination is detected, further wherein said urine analysis module comprises microfluidic biosensors selected from the group consisting of glucose, ketones, protein, pH, nitrites, leukocytes, bilirubin, and urobilinogen.

15. A method of diagnosing a medical ailment, the method comprising the steps of:
providing an AI healthcare bed comprising an AI powered diagnostic module, a body scanning module, a urine analysis module, a vital signs monitoring suite, and a blood analysis module;
performing full-body surface and subsurface scans with said body scanning module using imaging sensors selected from the group consisting of infrared sensors, ultrasonic sensors, and optical sensors;
autonomously collecting and analyzing with said urine analysis module urine samples from a user lying on the AI healthcare bed to detect biomarkers selected from the group consisting of kidney function, infections, hydration status, and metabolic disorders;
wherein said vital signs monitoring suite comprising one or more sensors selected from the group consisting of an ECG sensor, a body temperature sensor, a respiratory sensor, a blood pressure sensor, an SpO2 sensor;
wherein said blood analysis unit comprising optical sensors for monitoring glucose levels, hemoglobins, electrolytes, and blood biomarkers; and
further wherein said AI powered diagnostic module comprising an AI-powered processor for receiving in real-time information selected from the group consisting of physiological data, biochemical data, and behavioral data from one or more of said body scanning module, said urine analysis module, said vital signs monitoring suite, and said blood analysis unit.

16. The method of diagnosing a medical ailment of claim 15 further providing a display device comprising a touch screen device coupled to said AI powered diagnostic module, wherein said display device displays vital signs as visual feedback about a user lying on the AI healthcare bed.

17. A method of diagnosing a medical ailment and communicating with a healthcare provider, the method comprising the steps of:
providing an AI healthcare bed comprising an AI powered diagnostic module, a body scanning module, a urine analysis module, a vital signs monitoring suite, a blood analysis module, and a telemedicine module;
performing full-body surface and subsurface scans with said body scanning module using imaging sensors selected from the group consisting of infrared sensors, ultrasonic sensors, and optical sensors;
autonomously collecting and analyzing with said urine analysis module urine samples from a user lying on the AI healthcare bed to detect biomarkers selected from the group consisting of kidney function, infections, hydration status, and metabolic disorders;
wherein said vital signs monitoring suite comprising one or more sensors selected from the group consisting of an ECG sensor, a body temperature sensor, a respiratory sensor, a blood pressure sensor, an SpO2 sensor;
wherein said blood analysis unit comprising optical sensors for monitoring glucose levels, hemoglobins, electrolytes, and blood biomarkers;
wherein said AI powered diagnostic module comprising an AI-powered processor for receiving in real-time information selected from the group consisting of physiological data, biochemical data, and behavioral data from one or more of said body scanning module, said urine analysis module, said vital signs monitoring suite, and said blood analysis unit; and
initiating with said telemedicine module a remote consultation with a licensed healthcare provider, wherein said telemedicine module is configured to share real-time data from said AI powered diagnostic module.

18. The method of diagnosing a medical ailment and communicating with a healthcare provider of claim 17 further comprising a step of providing a display device comprising a touch screen device coupled to said AI powered diagnostic module, wherein said display device displays vital signs as visual feedback about a user lying on the AI healthcare bed.

19. The method of diagnosing a medical ailment and communicating with a healthcare provider of claim 18 further comprising a step of providing a wireless communication module configured to provide bi-directional data exchange with an external device selected from the group consisting of a telepath network, a remote server, and an electronic health record (EHR).

20. The method of diagnosing a medical ailment and communicating with a healthcare provider of claim 19, wherein said remote server comprises a patient database to store anonymized information selected from the group consisting of encrypted health records, vital signs, diagnostic history, and treatment compliance data of different users received from the AI healthcare bed; and
further comprising a doctor database for storing information selected from the group consisting of licensed healthcare providers, provider availability, provider specialties, and provider certifications, wherein said remote server uses said patient database and said doctor database to recommend one or more said licensed healthcare providers for the user to receive a telemedicine session.
